(19) United States
(12) Patent Application Publication
(10) Pub. No.: US 2006/0281093 A1
(43) Pub. Date: Dec. 14, 2006

(54) Erbb receptor methods and kits for monitoring chemotherapy resistance

(76) Inventor: Richard Kim, New York, NY (US)

Correspondence Address: JONES DAY, 222 EAST 41ST ST, NEW YORK, NY, 10017, US

(21) Appl. No.: 10/567,702

(22) PCT Filed: Aug. 5, 2004
(86) PCT No.: PCT/US04/25545

§ 371 Date: Aug. 21, 2006

Related U.S. Application Data
(60) Provisional application No. 60/492,759, filed on Aug. 5, 2003.

(51) Int. Cl. C12Q 1/68; G01N 33/574

(52) U.S. Cl. 435/6; 435/7.23

(57) ABSTRACT

The present invention relates to monitoring of ErbB receptor levels in methods and kits for determining the prognosis of cancer in a subject or improving the effectiveness of a cancer treatment. The invention also provides a method for predicting the recurrence of clinical signs of a cancer in a subject. In some embodiments, the invention provides methods for predicting the development of resistance to a chemotherapy regimen. In other embodiments, the invention provides methods for improving the effectiveness of a cancer treatment in a subject by monitoring levels of ErbB-2, ErbB-3 and/or ErbB-4. Preferably, the subject in the methods of the invention has been previously treated with a chemotherapy regimen for an ErbB-1 positive tumor.

This application claims priority to U.S. Provisional Application No. 60/492,759 filed Aug. 5, 2003 which is incorporated herein by reference in its entirety.

### 1. FIELD OF THE INVENTION

The present invention relates to methods and kits for monitoring ErbB receptor levels for determining the prognosis of cancer or improving the effectiveness of a cancer treatment in a subject. The invention also provides methods for predicting the recurrence of clinical signs of cancer in a subject. In some embodiments, the invention provides methods for predicting the development of resistance to a chemotherapy regimen. In other embodiments, the invention provides methods for improving the effectiveness of a cancer treatment in a subject by monitoring levels of ErbB-2, ErbB-3, ErbB-4 or a combination thereof. Preferably, the subject in the methods of the invention has been previously treated with a chemotherapy regimen for an ErbB-1 positive tumor.

### 2. BACKGROUND OF THE INVENTION

2.1 ErbB Receptor Family and Cell Signaling

Many cell surface molecules communicate information from the external milieu to the interior of the cell. This “sensing” is critical in multicellular organisms as the cells must function appropriately and respond in concert to the changing needs of the organism. One major family of cell surface sensors is the ErbB family, comprised of transmembrane receptors with intrinsic protein tyrosine kinase activity. The prototypical member of this family is the epidermal growth factor receptor (EGFR), also referred to as HER, human EGFR or ErbB-1. The EGFR was the first receptor described to possess tyrosine kinase activity and the first member of the ErbB receptor family to be cloned and sequenced (see review by Schlessinger, 2000, Cell 103: 211-225; Simon, 2000, Cell 103: 13-15).

The EGFR (ErbB-1) is one among four closely related receptors including Her2/neu (ErbB-2), HER-3 (ErbB-3), and HER-4 (ErbB-4). These receptors exist as inactive monomers in the cell membrane, and dimerize after ligand binding-induced activation. Dimerization may be homodimerization, or heterodimerization between EGFR and another member of the ErbB receptor family. After ligand binding, the tyrosine kinase intracellular domain of the ErbB receptor is activated. Next, autophosphorylation of the intracellular domain occurs, initiating a cascade of intracellular events. The ErbB receptor signaling pathway involves activation of ras and mitogen-activated protein kinase, which in turn activates several nuclear proteins, including Cyclin D1, a protein required for cell cycle progression from G1 to S phase (see review by Wells, 1999, Int. J. of Biochem, & Cell Biol. 31: 637-43).

Epidermal growth factor (EGF) is the prototypical ligand for ErbB-1 and is a member of a family of related growth factors, all of which bind ErbB-1, including transforming growth factor alpha (TGFα), amphireguline, heparin binding EGF, and betacellulin. By contrast, the ligand for ErbB-2 is undefined, while the ErbB-3 and ErbB-4 receptors serve as heregulin and neuregulin receptors, respectively (see review by Wells, 1999, Int. J. of Biochem, & Cell Biol. 31: 637-43).

2.2 Role of ErbB Receptor Family in Cancer Development and Progression

The EGFR or ErbB family of receptors is believed to play a crucial role in cancer pathogenesis, and it has been extensively reviewed in the scientific literature (see, Goustin et al., 1986, Cancer Res. 46: 1015-29; Aaranson, 1991, Science 254: 1146-53; Sedlacek, 2000, Drugs 59: 435-76; Wells, 1999, Int. J. of Biochem, & Cell Biol. 31: 637-43; Noonberg et al., 2000, Drugs 59: 753-67; Woodburn, 1999, Pharmacol. Ther. 82: 241-50; Olayioye et al., 2000, EMBO J. 19: 3159-67; Tang, C. K. & Lipmann, M. E. in Hormones and Signaling, ed. O'Malley, B. W., 113-165, Academic Press, San Diego). ErbB receptors have been implicated in numerous types of cancers, for example, non-small cell lung cancer, breast, head and neck, prostate, bladder, ovarian, colorectal and glioblastomas. ErbB receptors were first implicated in cancer when the avian erythroblastosis tumor virus was found to encode an aberrant form of ErbB-1. ErbB receptors have also been implicated in cellular proliferation, apoptosis, differentiation, angiogenesis, motility and invasion. Dysregulation of ErbB signaling in cancer can occur by various mechanisms, including gene amplification and ErbB mutations that increase receptor transcription, translation or protein stability. Therapeutic blockade of ErbB-1 signaling is believed to be beneficial in treatment of patients with cancer (see, e.g., Woodburn, 1999, Pharmacol. Ther. 82: 241-50).

A variety of methods for inhibiting the action of EGFR or its stimulatory ligands have been investigated, and a number of promising therapeutics have been developed including for example, enzyme inhibitors, antibodies, antisense oligonucleotides and fusion proteins. Initial studies indicate a promising potential for EGFR inhibition in cancer therapy (for reviews see Sedlacek, 2000, Drugs 59: 435-76; Wells, 1999, Int. J. of Biochem, & Cell Biol. 31: 637-43; Noonberg et al., 2000, Drugs 59: 753-67; Woodburn, 1999, Pharmacol. Ther. 82: 241-50.)

One promising approach to the treatment of ErbB-related cancers has been the development of EGFR-specific chemotherapeutic agents, some of which are in clinical trials (for example IMC-C225 and ZD1839). Many chemotherapeutic agents, however, become limited in their effectiveness due to the development of resistance. It remains unknown if targeting one ErbB receptor may eventually lead to upregulation of other ErbB receptors, leading to clinical development of resistance (DeBono et al., 2002, Trends in Mol. Med. 8:19-26). Accordingly, there is a need in the art for the development of prognostic methods for evaluation of subjects in remission, and for improving the effectiveness of cancer treatment in subjects with active ErbB-related cancers.

### 3. SUMMARY OF TH INVENTION

The present invention provides methods for influencing the disease course in a subject, preferably a human, with cancer or a history of cancer. The invention also provides methods for prognosis and for determining a course of treatment for a subject with cancer so that the cancer treatment in the subject is improved. The methods of the invention are particularly useful in a subject with an ErbB-related cancer, i.e., a cancer associated with an aberrant expression and/or activity of an ErbB receptor protein. In particular, the invention is based, in part, on the discovery that monitoring a level of one or more ErbB receptors, including, but not limited to, any one or more of ErbB-2, ErbB-3, and ErbB-4, in a subject with an ErbB-1 positive tumor provides the clinician with a prognostic measure of the cancer in the subject. The invention encompasses measuring the level of at least one ErbB receptor or a combination of two or more ErbB receptors in one or more samples from the subject. In a specific embodiment, the invention encompasses measuring a level of ErbB-2, ErbB-3, or ErbB-4 or a combination thereof. In another specific embodiment, the invention encompasses measuring a level of ErbB-3 or ErbB-4 or both. In one embodiment, the invention encompasses measuring a level of ErbB-2 or ErbB-3 or both. In another embodiment, the invention encompasses measuring a level of ErbB-2 or ErbB-4 or both. Monitoring a level of an ErbB receptor may include, but is not limited to, monitoring ErbB receptor activity, receptor protein abundance and ErbB receptor mRNA expression profiles.

In one embodiment, the invention provides a method for predicting the recurrence of clinical signs of a cancer in a subject. In a preferred embodiment, the subject is human. In a most preferred embodiment, the subject has been previously treated with a therapy regimen, e.g., chemotherapy, radiotherapy, for an ErbB-1 positive tumor. Chemotherapy and radiotherapy regimens for the treatment of a cancer, particularly an ErbB-1 positive tumor, are well known in the art, and examples are disclosed herein. In a specific embodiment the method for predicting the recurrence of clinical signs of a cancer in a subject comprises measuring a level of at least one ErbB receptor in a sample obtained from the subject during a period of remission and determining whether the subject is at an increased risk for the recurrence of clinical signs of the cancer from the level measured. In another specific embodiment, the method for predicting the recurrence of clinical signs of a cancer in a subject comprises: measuring a level of at least one ErbB receptor in a sample obtained from the subject during a period of remission; and comparing the level measured to a standard level, wherein elevation of the measured level of at least one ErbB receptor relative to the standard level indicates that the subject is at an increased risk for the recurrence of clinical signs of the cancer. In one embodiment, the level of the ErbB receptor is serially monitored.

In other embodiments, the invention encompasses a method for determining the prognosis of a cancer in a subject. Preferably, the subject is human, and most preferably the subject has been previously treated with a therapy regimen, e.g., chemotherapy, radiotherapy, for an ErbB-1 positive tumor. In a specific embodiment, the method for determining the prognosis of a cancer in a subject comprises: measuring a level of at least one ErbB receptor in a sample obtained from the subject during a period of remission; and comparing the level measured to a standard level, wherein elevation of the measured level of at least one ErbB receptor relative to the standard level indicates that the subject is at an increased risk for metastasis, recurrence or relapse of the cancer. In one embodiment, the level of the ErbB receptor is serially monitored.

In other embodiments, the invention encompasses a method for predicting the development of resistance to a therapy regimen, e.g., chemotherapy, radiotherapy, in a subject, preferably a human, most preferably a subject which has been previously treated with a chemotherapy regimen for an ErbB-1 positive tumor. In a specific embodiment, the method for predicting the development of resistance to a chemotherapy regimen in a subject comprises: measuring a level of at least one ErbB receptor in a sample obtained from the subject during a period of remission; and comparing the level measured to a standard level, wherein elevation of the measured level of at least one ErbB receptor relative to the standard level indicates that the subject is at an increased risk for development of resistance to the chemotherapy regimen. In one embodiment, the level of the ErbB receptor is serially monitored.

In other embodiments, the invention encompasses a method for improving the effectiveness of cancer treatment in a subject with cancer, preferably a human subject. In a specific embodiment, the method comprises: treating the subject with a treatment regimen so as to achieve remission; measuring a level of at least one ErbB receptor in a sample obtained from the subject during a period of remission; and comparing the level measured to a standard level, wherein elevation of the measured level of one ErbB receptor relative to the standard level indicates that the subject is in need of additional treatment. In one embodiment, the level of the ErbB receptor is serially monitored.

In various embodiments of the methods of the invention, the ErbB receptor level that is measured is not any one or more of ErbB-1, ErbB-2, ErbB-3, or ErbB-4.

In various embodiments disclosed herein, the invention encompasses obtaining at least one sample, including but not limited to a biological fluid (e.g., blood, urine), or a tissue sample, from at least one subject with cancer or a history of cancer. The sample may be obtained using any methodology known to one skilled in the art. The methods of the invention are particularly useful when the cancer is any cancer involving an overexpression and/or aberrant expression of at least one ErbB receptor. In particularly preferred embodiments of the invention, the cancer is selected from the group consisting of non-small cell lung cancer, breast cancer, head and neck cancer, prostate cancer, bladder cancer, ovarian cancer, colorectal cancer, and glioblastoma.

In a specific embodiment, the invention encompasses measuring at least one ErbB receptor level for any of the above-mentioned cancers comprising using an ErbB receptor probe. The ErbB receptor probe may include but is not limited to an antibody or a fragment thereof, a nucleic acid, a protein and a small molecule. In one specific embodiment, the protein is an ErbB receptor ligand or a fragment thereof. In another specific embodiment, the probe is an anti-ErbB receptor antibody that immunospecifically binds an ErbB receptor, preferably a monoclonal antibody, or an immunospecific fragment or derivative thereof. In yet another specific embodiment, the probe is a nucleic acid.

The invention further relates to a kit comprising: (a) at least one reagent selected from the group consisting of an anti-ErbB receptor antibody or an immunospecific fragment thereof, a nucleic acid probe capable of specifically hybridizing to an ErbB receptor mRNA, and a pair of nucleic acid primers capable of PCR amplification of at least a portion of an ErbB receptor nucleic acid; and (b) printed instructions for use in measuring a level of at least one ErbB receptor in a subject for a purpose of this invention. In a particular embodiment the kit further comprises a predetermined amount of a purified ErbB receptor protein or nucleic acid for use as a standard or control. In another specific embodiment, the reagent in the kit is labeled with a detectable marker. The detectable marker may include but is not limited to a chemiluminescent, enzymatic, fluorescent or radioactive label.

In a specific embodiment for any of the methods and kits of the invention, measuring a level of an ErbB receptor in a sample comprises contacting the sample with an antibody or a fragment thereof that is immunospecific for an ErbB receptor; and quantitating any binding that has occurred between the antibody or a fragment thereof and an ErbB receptor in the sample. In another specific embodiment, measuring a level of an ErbB receptor in a sample comprises contacting the sample with a nucleic acid that hybridizes specifically to an ErbB receptor mRNA and quantitating any hybridization that has occurred between the nucleic acid probe and the mRNA in the sample.

In another embodiment, measuring a level of an ErbB receptor in a sample comprises quantitating ErbB receptor activity in the sample (e.g., via measuring receptor tyrosine kinase activity by any method known in the art).

### 4. DETAILED DESCRIPTION OF THE INVENTION

The present invention provides methods and kits for cancer prognosis and therapy optimization in a subject. The methods and kits of the invention are particularly useful for cancers in remission which may display an elevated level of expression and/or activity of an ErbB receptor, including but not limited to ErbB-1, ErbB-2, ErbB-3, and ErbB-4, as an early sign of chemotherapy resistance or exit from remission. Particular cancers amenable to the methods of the invention include but are not limited to, non-small cell lung cancer, breast cancer, head and neck cancer, prostate cancer, bladder cancer, ovarian cancer, colorectal cancer, and glioblastoma.

In a particular embodiment, the invention provides a method for predicting the recurrence of clinical signs of a cancer in a subject, preferably human, comprising serially monitoring a level of at least one ErbB receptor in a sample obtained from the subject during a period of remission; and comparing the level measured to a standard level, wherein elevation of the measured level relative to the standard level indicates that the subject is at an increased risk for the recurrence of clinical signs of the cancer. In a specific embodiment, the subject has been previously treated with a chemotherapy regimen for an ErbB-1 positive tumor.

As used herein, “clinical signs of cancer” refers to any sign or indication of the existence of cancer in a subject, which sign or indication would be well known to the skilled artisan (e.g., oncologist, nurse practitioner). The clinical signs of cancer may refer to any symptom known to be associated with the cancer. Clinical signs of some cancers include, for example, chronic pain, nausea, vomiting, abnormal taste sensation, constipation, urinary symptoms (e.g., bladder spasm), respiratory symptoms, skin problems (e.g., pruritus, hair loss), or fever, among others.

As used herein, “remission” refers to a period during which the symptoms of a cancer have been reduced or eliminated, as remission is ordinarily defined in the oncology art.

As used herein “serially monitoring” a level of an ErbB receptor in a sample, refers to measuring a level of an ErbB receptor in a sample more than once, e.g., quarterly, bimonthly, monthly, biweekly, weekly, every three days or daily. Serial monitoring of a level includes periodically measuring a level of an ErbB receptor at regular intervals as deemed necessary by the skilled artisan.

The term “standard level” as used herein refers to a baseline amount of an ErbB receptor level as determined in one or more normal subjects. For example, a baseline may be obtained from at least one subject and preferably is obtained from an average of subjects (e.g., n=2 to 100 or more), wherein the subject or subjects have no prior history of cancer. In the present invention, the measurement of an ErbB receptor level may be carried out using an ErbB receptor probe or an ErbB receptor activity assay.

As used herein, “elevation” of a measured level of an ErbB receptor relative to a standard level means that the amount or concentration of an ErbB receptor in a sample is sufficiently greater in a subject relative to the standard to be detected by any method known in the art or to be developed in the future for measuring an ErbB receptor level. For example, elevation of the measured level relative to a standard level may be any statistically significant elevation which is detectable. Such an elevation may include, but is not limited to, about a 1%, about a 10%, about a 20%, about a 40%, about an 80%, about a 2-fold, about a 4-fold, about an 8-fold, about a 20-fold, or about a 100-fold elevation, or more, relative to the standard. The term “about” as used herein, refers to a numerical value plus or minus 10% of the numerical value.

As used herein, reference to “measuring a level of an ErbB receptor” in a method of the invention means measuring the ErbB receptor level or any proxy for an ErbB receptor level. Such proxies may include, but are not limited to, ErbB receptor tyrosine kinase activity assays. A level of an ErbB receptor may correspond to the abundance of full-length ErbB receptor protein. Alternatively, a level of an ErbB receptor may correspond to abundance of a fragment of an ErbB receptor protein. A level of an ErbB receptor can be determined by measuring the abundance of nucleic acids (or sequences complementary thereto) that encode all or a portion of an ErbB receptor. In a preferred embodiment, the abundance of mRNA encoding an ErbB receptor is determined using quantitative PCR.

As used herein, a probe with which the amount or concentration an ErbB receptor can be determined, includes but is not limited to a nucleic acid, a protein (e.g., an antibody), or a small molecule (e.g., OS1-774, OSI Pharmaceuticals, Inc./Genentech, Inc.). In a specific embodiment, the probe is an ErbB receptor ligand (e.g., neuregulin) or a fragment thereof that specifically binds the ErbB receptor. In another embodiment, the probe is an antibody that immunospecifically binds to ErbB receptor, such as e.g., a monoclonal antibody or a binding fragment thereof.

In another embodiment, the invention encompasses a method for determining the prognosis of a cancer in a subject, preferably a subject that has been previously treated with a chemotherapy regimen for an ErbB-1 positive tumor, comprising: serially monitoring a level of at least one ErbB receptor in a sample obtained from the subject during a period of remission; and comparing the level to a standard level, wherein elevation of the measured level of at least one ErbB receptor relative to the standard level indicates that the subject is at an increased risk for metastasis, recurrence or relapse of the cancer.

In another embodiment, the invention encompasses a method for predicting the development of resistance to a chemotherapy regimen in a subject, which subject has preferably been treated with a chemotherapy regimen for an ErbB-1 positive tumor, comprising: serially monitoring a level of at least one ErbB receptor in a sample obtained from the subject during a period of remission; and comparing the level measured to a standard level, wherein elevation of the measured level relative to the standard level indicates that the subject is at an increased risk for development of resistance to the chemotherapy regimen.

The chemotherapy regimen to which the subject has become resistant may include any chemotherapy treatment known in the art for treatment of cancer, particularly a cancer associated with aberrant expression and/or activity of an ErbB-1 receptor, including but not limited to, treatment with chemotherapeutic agents directed at the ErbB signaling pathway such as, e.g., IMC-225 (an antibody that binds ErbB-1 and is believed to block EGF-induced autophosphorylation; Imclone Systems, New York, N.Y., USA), or ZD1839 (a quinizalone derivative which is a selective, reversible inhibitor of ErbB-1 tyrosine kinase activity, AstraZeneca PLC).

Non-limiting examples of chemotherapeutic agents known in the art are methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, Gleevec™ (imatinib mesylate), herbimycin A, genistein, erbstatin, and lavendustin A. In a preferred embodiment, the chemotherapeutic agent is Gleevec™ (imatinib mesylate). Further examples of chemotherapeutic agents may be found in standard texts. See e.g., Manual of Clinical Oncology, Dennis A. Casciato and Barry B. Lowitz, ed., 4^{th} edition, Jul. 15, 2000, Little, Brown and Company, U.S.

Radiotherapy is also well known in the art. See, e.g., DeVita, Vincent, Hellman, Samuel, and Rosenberg, eds., Cancer: Principles and Practice of Oncology, 6^{th} ed., Lippincott Williams & Wilkins, July 2001, which is incorporated herein by reference in its entirety.

The invention further encompasses a method for improving the effectiveness of cancer treatment in a subject with cancer, comprising: treating the subject with a treatment regimen so as to achieve remission; serially monitoring a level of at least one ErbB receptor in a sample obtained from the subject during a period of remission; and comparing the level measured to a standard level, wherein elevation of the measured level of at least one ErbB receptor relative to the standard level indicates that the subject is in need of additional treatment.

In a further embodiment, the antibody or other probe is labeled with a detectable marker. In a specific embodiment, the detectable marker is a chemiluminescent, enzymatic, fluorescent, or radioactive label.

In a specific embodiment for any of the methods and kits of the invention, the step of measuring a level of an ErbB receptor in a sample comprises contacting the sample with an antibody or a fragment thereof that is immunospecific for an ErbB receptor; and quantitating any binding that has occurred between the antibody or a fragment thereof and an ErbB receptor in the sample. In another specific embodiment, the step of measuring a level of an ErbB receptor in a sample comprises contacting the sample with a nucleic acid that hybridizes specifically to an ErbB receptor mRNA and quantitating any hybridization that has occurred between the nucleic acid probe and the mRNA in the sample.

The methods of the invention may be used to measure a level of any nucleic acid encoding an ErbB receptor protein, including but not limited to, ErbB-1, ErbB-2, ErbB-3, and ErbB-4. For example, the methods of the invention may use at least a portion of (i) the nucleotide sequence of human ErbB-1, e.g., as derived from placental and A431 carcinoma cells (Ulrich et al., 1984, Nature 309:418-425); (ii) the nucleotide sequence of human ErbB-2, e.g., as cloned from a human fetal DNA library by Coussens et al. (1985, Science 230: 1132-9); (iii) the nucleotide sequences of any one or more of exons 1-7 of human ErbB-2 with GENBANK accession numbers AH001455, M11762, M11763, M11764, M11765, M11766, M11767, respectively; (iv) the nucleotide sequence of human ErbB-3, e.g., ErbB-3 isolated from a human carcinoma cell line (Plowman et al. 1990, Proc. Natl. Acad. Sci. USA. 87: 4905-9); or (v) the nucleotide sequence of gallus ErbB-4 with GENBANK accession number AF041792. All of the nucleotide sequences cited in these references are incorporated herein by reference in their entireties.

The methods of the invention may also be used to measure a level of one or more ErbB receptor proteins, including but not limited to, ErbB-1, ErbB-2, ErbB-3, and ErbB-4. For example, the methods of the invention may measure ErbB receptor protein abundance by polyacrylamide gel electrophoresis (PAGE) or enzyme-linked immunosorbent assay (ELISA) or any other standard method known in the art for quantitation of protein abundance.

In another embodiment, the invention provides a kit comprising: (a) at least one reagent capable of quantitating an ErbB receptor level; and (b) printed instructions for using the reagent in a method of the invention. ErbB receptor levels may be quantitated, e.g., using a reagent selected from the group consisting of an anti-ErbB receptor antibody, a nucleic acid probe capable of hybridizing with an ErbB receptor mRNA, and a pair of nucleic acid primers capable of priming amplification of at least a portion of an ErbB receptor nucleic acid. The instructions may describe one or more of the various embodiments of the present invention. For example, the instructions may detail an ErbB receptor activity assay. In another specific embodiment, the kit further comprises a predetermined amount of a purified ErbB receptor protein or nucleic acid encoding an ErbB receptor or a fragment thereof sufficient for use as a standard or control. In a further specific embodiment, the reagent in the kit is labeled with a detectable marker. In a specific embodiment, the detectable marker is a chemiluminescent, enzymatic, fluorescent, or radioactive label.

4.1 Cancer Prognostic Methods

The present invention provides various methods and kits for monitoring the level of an ErbB receptor selected from ErbB-1, ErbB-2, ErbB-3, and ErbB-4, using any method available in the art, to improve cancer therapy and/or prognosis in a subject. Particularly, ErbB receptor monitoring is useful in: (i) predicting the recurrence of clinical signs of a cancer in a subject, e.g., a subject with an ErbB-1 positive tumor, who has previously been treated with a chemotherapy regimen; (ii) determining the prognosis of a cancer in a subject who has been previously treated with a chemotherapy regimen; (iii) predicting the development of resistance to a chemotherapy regimen in a subject with cancer; and (iv) improving the effectiveness of a cancer treatment in a subject with cancer.

Any method known in the art for detecting and/or quantitating an ErbB receptor level may be used in the methods and kits of the invention, a number of which are exemplified herein. Particularly preferred are methods known in the art for detecting and/or quantitating an ErbB receptor activity or an ErbB receptor related activity, e.g., ErbB receptor tyrosine kinase activity.

In some embodiments, the invention encompasses measuring an ErbB receptor activity or an ErbB receptor related activity including but not limited to, measuring a level of an ErbB receptor tyrosine kinase activity or measuring an activity of one or more downstream effectors of an ErbB receptor signaling casade. Measuring an ErbB receptor activity or an ErbB receptor related activity can be done using any of the methods disclosed herein or any standard method known to one skilled in the art.

In other embodiments, the invention encompasses quantitation of a nucleic acid encoding an ErbB receptor in a sample obtained from a subject using methods disclosed herein or any standard method known in the art.

In yet other embodiments, the invention encompasses quantitation of ErbB-1, ErbB-2, ErbB-3, ErbB-4 or a combination thereof in a sample obtained from a subject with cancer. Any method known in the art for the detection and quantitation of an ErbB receptor protein is encompassed within the present invention.

4.2 Samples Used in the Methods of the Invention

A sample for the prognostic methods of the invention encompasses any sample that can be obtained preferably by a non-invasive technique from a subject. Preferably, the methods for obtaining a sample from a subject are not time consuming. A sample for the purposes of the invention may include but is not limited to, a biological fluid such as serum, plasma, urine, or blood; a tissue sample; or a tissue extract. Such samples may be obtained by any standard method known in the art, e.g., a finger stick blood sample, a buccal swab, a biopsy, etc.

In preferred embodiments, a sample for the methods of the invention is a blood or serum sample obtained periodically from the subject. The sample used in accordance with the methods of the invention need not be obtained from the particular tissue from which the tumor originated. Although not intending to be bound by a particular mechanism of action, given that ErbB receptors are ubiquitously expressed, when therapy, e.g., chemotherapy, is targeted at an ErbB receptor, essentially all ErbB receptors are targeted in the body. Therefore, once resistance to the ErbB-directed therapy is developed, it would be detectable throughout the body and not just from the particular tissue from which the tumor originated.

The invention encompasses use of any tissue sampling or biopsy technique known in the art for obtaining a sample from a subject with cancer. In some embodiments, when the subject has breast cancer or a history of breast cancer, any method for obtaining breast tissue known to one skilled in the art can be used, including but not limited to, core biopsies and fine-needle aspirations (see, e.g. Lawrence et al., 2001, J. Clin. Oncol. 19: 2754-63; Fabian et al., 1993, J. Cell. Biochem 17G: 153-160; Boerner et al., 1999, Cancer 87(1): 19-24; Rotten et al., 1993, Eur. J. Obstet. Gynecol. Reprod. Biol. 49(3): 175-86; which are incorporated herein by reference in their entirety). In other embodiments, the invention encompasses lavage and nipple aspiration of breast ductal fluids to obtain a breast tissue sample from a subject with cancer. An exemplary method for lavage and nipple aspiration of breast ductal fluids is presented in Klein et al., (2002, Environmental and Molecular Mutagenesis 39: 127-33), which is incorporated herein by reference in its entirety.

In other embodiments, when the subject has prostate cancer or a history of prostate cancer, any biopsy technique known in the art, including but not limited to needle biopsy and transrectal aspiration biopsy, can be used in the methods of the invention. See, e.g., Kaufman et al., 1982, Urology 19(6): 587-91, which is incorporated herein by reference in its entirety.

In some embodiments, when the subject has colorectal cancer, any biopsy or tissue sampling technique known in the art, including but not limited to needle aspiration and solid biopsy, are within the scope of the invention. See, e.g., Greenebaum et al., 1984, Am. J. Clin. Pathol. 82(5): 559-64; which is incorporated herein by reference in its entirety.

In the case of lung cancer, the invention encompasses the use of any tissue sampling and biopsy methods known in the art, including but not limited to, fine needle aspirations, EUS-guided fine needle aspirations, bronchial biopsy, transesophogeal biopsy, and broncholaveolar lavage. See, e.g., Devereaux et al., 2002, Gastorintest. Endosc. 56: 397-401; Rosell et al., 1998, Eur. Respir. J. 12(6): 1415-8; Hunerbein et al., 1998, J. Thorac. Cardiovasc. Surge. 116(4): 554-9; Kvale, 1996, Chest Surg. Clin. N. Am. 6: 205-22, all of which are incorporated herein by reference in their entirety.

4.3 Methods of Quantitating ErbB Receptor Levels

ErbB receptor levels may be quantitated in the methods and kits of the invention by measuring, e.g., receptor activity, nucleic acid abundance, or protein abundance.

4.3.1 Assays for ErbB Receptor Activity

An ErbB receptor triggers numerous downstream signaling pathways upon ligand binding subsequent to the activation of the tyrosine kinase domain by autophosphorylation. There are five sites of autophosphorylation, at amino acid positions 992, 1068, 1086, 1148, and 1173, in the intracellular kinase domain of an ErbB receptor (excluding ErbB-3, since the sequence of the ErbB-3 catalytic domain suggests that this receptor does not have protein tyrosine kinase catalytic activity; see Carraway and Cantley, 1994, Cell 78: 5-8; Schlessinger, 2000, Cell 103: 211-225). Tyrosine autophosporylation of the ErbB receptor leads to the recruitment and activation of a variety of signaling proteins, specifically signaling proteins comprising PTB and SH2 domains, which in turn lead to the recruitment of a family of proteins containing other docking sites, including but not limited to PH domains, SH3 domains, WW domains, PDZ domains, and FYVE domains, which mediate ErbB receptor activation (for a review see, Schlessinger, 2000, Cell 103: 211-225).

The ras-MAP kinase cascade is activated by the ErbB receptor signaling mechanism (see Wells, 1999, The Int. J. of Biochem. & Cell Biol. 31: 637-43). Both the PLCγ-mediated pathway and the ras-mediated pathway relay signals introduced via the ErbB receptor family. PLCγ is rapidly recruited to an activated ErbB receptor via binding of its SH2 domain to phosphorylated tyrosine sites of the ErbB intracellular kinase domain; upon activation, PLCγ hyrdolyzes its substrate, PtdIns(4,5)P₂, to form two second messengers, DAG and Ins(1,4,5)P₃, which in turn bind specific intracellular receptors and lead to calcium release.

The invention encompasses measuring a level of an ErbB receptor related activity and/or ErbB receptor activity, including but not limited to measuring a level of an ErbB receptor tyrosine kinase activity and measuring the activity of one or more downstream effectors of an ErbB receptor signaling cascade, such as PLCγ, ras, MAP kinase, PKC, etc. The invention encompasses methods of measuring one or more ErbB receptor mediated biological responses using standard assays known to one skilled in the art, for example, measuring calcium mobilization by flow cytometry, measuring phosphorylation of the tyrosine kinase domain of an ErbB receptor, and measuring the phosphorylation and activation of MAPK. In a specific embodiment, the invention encompasses methods of measuring the activation of one or more downstream signaling molecules of the ErbB receptor signaling pathway.

In some embodiments, the assays of the present invention may include in vitro kinase assays which measure the amount of tyrosine phosphorylation of an ErbB receptor in a sample obtained from a subject and comparing the amount of tyrosine phosphorylation of the ErbB receptor relative to a standard level. For example, but not by way of limitation, these assays may involve immunoprecipitation of an ErbB receptor from a sample using methods known to one skilled in the art using any of the ErbB specific antibodies disclosed herein in Section 4.9 (or commercially available such as those from Upstate USA, Inc. Charlottesville, Va.; see http://www.upstate.com), including but not limited to, anti-phospho-ErbB-2 polyclonal antibody (Y1428), anti-ErbB-2 antibody, anti-ErbB-3 clone 2F12, anti-ErbB-3 clone H3.105.5, anti-ErbB-4 monoclonal and polyclonal antibody, and measuring the ErbB receptor autophosphorylation activity of the immunoprecipitated kinase. The phosphorylation of the ErbB receptor may be determined for example using commercially available anti-phosphotryosine antibodies (e.g., Upstate USA, Inc.).

In alternative embodiments, a component of the ErbB receptor signaling cascade may be immunoprecipitated from the sample and its kinase activity may be measured using a substrate of the kinase. For example, in one embodiment MAP kinase may be immunoprecipitated from the sample and the kinase activity may be measured using a known substrate of the kinase, e.g., myelin basic protein, transcription factors such as AFT-2, CHOP, HSP27, and MAX. The activity of the MAP kinase may be determined as a measurement of the phosphorylated state of the substrate. Substrates and antibodies to MAP kinase are known in the art and commercially available for example from Upstate USA, Inc.

In yet other embodiments, a component of the ErbB receptor signaling cascade that gets recruited upon ErbB activation may be immunoprecipated from the sample using antibodies available for the components, and the amount of recruited component may be compared to a standard level. Such components are well known in the art. For example, but not by way of limitation, these assays may involve immunoprecipitation of a component from the ErbB receptor signaling cascade, e.g., Shc, Grb-2, etc., from a sample using methods known to one skilled in the art using any of the antibodies known in the art against the component (or commercially available such as those from Upstate USA, Inc.), and the amount of the component may be compared to a standard level.

4.3.2 Detection of Nucleic Acid Molecules

The methods and kits of the invention encompass detection and/or quantitation of a nucleic acid sequence encoding an ErbB receptor in a sample obtained from a subject. In certain embodiments, the invention provides methods for amplifying a specific ErbB receptor nucleic acid sequence in a sample obtained from a subject with cancer, and detecting and/or quantitating the same. Nucleic acids encoding ErbB receptors are well known in the art. See, e.g., Section 4.7 below.

The methods and kits of the invention may use any nucleic acid amplification or detection method known to one skilled in the art, such as those described in U.S. Pat. Nos. 5,525,462; 6,528,632; 6,344,317; 6,114,117; 6,127,120; 6,448,001; all of which are incorporated herein by reference in their entirety.

In some embodiments, the nucleic acid encoding an ErbB receptor is amplified by PCR amplification using methodologies known to one skilled in the art. One of skill in the art will recognize, however, that amplification of target sequences (i.e., nucleic acid sequences encoding an ErbB receptor) in a sample obtained from a subject with cancer can be accomplished by any known method, such as ligase chain reaction (LCR), QP-replicase amplification, transcription amplification, and self-sustained sequence replication, each of which provides sufficient amplification. The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; which is incorporated herein by reference in its entirety). Also see U.S. Pat. No. 4,683,202; which is incorporated herein by reference in its entirety. PCR reagents and protocols are also available from commercial vendors, such as Roche Molecular Systems.

The invention encompasses methods to determine quantitative and/or qualitative levels of expression of an ErbB receptor. Any technique known in the art for measuring the expression of an ErbB receptor is within the scope of the invention, including but not limited, to quantitative and/or semi-quantitative RT PCR and Northern blot analysis.

In some embodiments, the invention encompasses detecting and/or quantitating an ErbB receptor nucleic acid using fluorescence in situ hybridization (FISH) in a sample, preferably a tissue sample, obtained from a subject with cancer in accordance with the methods of the invention. FISH is a common methodology used in the art, especially in the detection of specific chromosomal aberrations in tumor cells, for example, to aid in diagnosis and tumor staging. As applied in the methods of the invention, it can also be used as a method for detection and/or quantitation of an ErbB receptor nucleic acid. For a review of FISH methodology, see, e.g., Weier et al., 2002, Expert Rev. Mol. Diagn. 2(2): 109-119; Trask et al., 1991, Trends Genet. 7(5): 149-154; and Tkachuk et al., 1991, Genet. Anal. Tech Appl. 8: 676-74; all of which are incorporated herein by reference in their entirety.

The invention encompasses measuring naturally occurring ErbB receptor transcripts and variants thereof as well as non-naturally occurring variants thereof. For the prognosis of cancer in a subject using the methods of the invention, the ErbB receptor transcript is preferably a naturally occurring ErbB receptor transcript.

Thus, the invention relates to methods of prognosis of a cancer in a subject by measuring the expression of an ErbB receptor transcript in a subject. For example, the increased level of mRNA encoding an ErbB receptor, as compared to a standard, e.g., a non-cancerous sample, would indicate the increased risk of developing cancer in said subject. In another embodiment, the increased level of mRNA encoding an ErbB receptor as compared to a standard would indicate the risk of metastasis of the cancer in said subject or the likelihood of a poor prognosis in said subject.

In one embodiment, the invention encompasses isolating RNA from a sample obtained from a subject with cancer, and testing the RNA utilizing hybridization or PCR techniques as described above for determining the level of an ErbB receptor. In another embodiment, the invention encompasses synthesizing cDNA from the isolated RNA by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the ErbB receptor nucleic acid reagents described in Section 4.7. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

In alternative embodiments, standard Northern analysis techniques known to one skilled in the art can be performed on a sample obtained from a subject with cancer. The preferred length of a probe used in Northern analysis is 9-50 nucleotides. Utilizing such techniques, quantitative as well as size related differences among ErbB receptor transcripts can also be detected.

In alternative embodiments, the invention encompasses gene expression assays in situ, i.e., directly upon tissue sections (fixed and/or frozen) of patient tissue obtained from biopsies or resections, such that no nucleic acid purification is necessary. Nucleic acid reagents such as those described in Section 4.7 may be used as probes and/or primers for such in situ procedures (see, e.g., Nuovo, G. J., 1992, PCR In Situ Hybridization: Protocols And Applications, Raven Press, NY, which is incorporated herein by reference in its entirety).

The target ErbB receptor nucleic acids of the invention can also be detected using other standard techniques well known to those of skill in the art. Although the detection step is typically preceded by an amplification step, amplification is not necessarily required in the methods of the invention. For instance, the ErbB receptor nucleic acids can be identified by size fractionation (e.g., gel electrophoresis). The presence of different or additional bands in the sample as compared to the control is an indication of the presence of target nucleic acids of the invention. Alternatively, the target ErbB receptor nucleic acids can be identified by sequencing according to well known techniques. In alternative embodiments, oligonucleotide probes specific to the target ErbB receptor nucleic acids can be used to detect the presence of specific fragments.

Sequence-specific probe hybridization is a well known method of detecting desired nucleic acids in a sample comprising a biological fluid or tissue sample and is within the scope of the present invention. Briefly, under sufficiently stringent hybridization conditions, the probes hybridize specifically only to substantially complementary sequences. The stringency of the hybridization conditions can be relaxed to tolerate varying amounts of sequence mismatch. If the target is first amplified, detection of the amplified product utilizes this sequence-specific hybridization to insure detection of only the correct amplified target, thereby decreasing the chance of a false positive caused by the presence of homologous sequences from related organisms or other contaminating sequences.

A number of hybridization formats well known in the art, including but not limited to solution phase, solid phase, mixed phase, or in situ hybridization assays are encompassed within the nucleic acid detection methods of the invention. In solution (or liquid) phase hybridizations, both the target nucleic acid and the probe or primer are free to interact in the reaction mixture. In solid phase hybridization assays, either the target or probes are linked to a solid support where they are available for hybridization with complementary nucleic acids in solution. Exemplary solid phase formats include Southern hybridizations, dot blots, and the like. The following articles provide an overview of the various hybridization assay formats, all of which are incorporated herein by reference in their entirety: Singer et al., 1986 Biotechniques 4: 230; Haase et al., 1984, Methods in Virology, Vol. VII, pp. 189-226; Wilkinson, In Situ Hybridization, D. G. Wilkinson ed., IRL Press, Oxford University Press, Oxford; and Nucleic Acid Hybridization: A Practical Approach, Hames, B. D. and Higgins, S. J., eds., IRL Press (1987).

The invention encompasses homogenous based hybridization assays as well as heterogeneous based assays for detection and/or quantitation of ErbB receptor nucleic acid sequences in accordance with the methods of the invention. Heterogeneous based assays depend on the ability to separate hybridized from non-hybridized nucleic acids. One such assay involves immobilization of either the target or probe nucleic acid on a solid support so that non-hybridized nucleic acids which remain in the liquid phase can be easily separated after completion of the hybridization reaction (see, e.g., Southern, 1975, J. Mol. Biol. 98: 503-517; which is incorporated herein by reference in its entirety). In comparison, homogeneous assays depend on other means for distinguishing between hybridized and non-hybridized nucleic acids. Because homogeneous assays do not require a separation step, they are generally considered to be more desirable. One such homogeneous assay relies on the use of a label attached to a probe nucleic acid that is only capable of generating a signal when the target is hybridized to the probe (see, e.g., Nelson, et al., 1992, Nonisotopic DNA Probe Techniques, Academic Press, New York, N.Y., pages 274-310; which is incorporated herein by reference in its entirety).

The invention encompasses any method known in the art for enhancing the sensitivity of the detectable signal in such assays, including but not limited to the use of cyclic probe technology (Bakkaoui et al., 1996, BioTechniques 20: 240-8, which is incorporated herein by reference in its entirety); and the use of branched probes (Urdea et al., 1993, Clin. Chem. 39: 725-6; which is incorporated herein by reference in its entirety).

The hybridization complexes are detected according to well known techniques in the art. Nucleic acid probes capable of specifically hybridizing to a target can be labeled by any one of several methods typically used to detect the presence of hybridized nucleic acids. One common method of detection is the use of autoradiography, using probes labeled with ³H, ¹²⁵I, ³⁵S, ¹⁴C, or ³²P, or the like. The choice of radioactive isotope depends on research preferences due to ease of synthesis, stability, and half lives of the selected isotopes. Other labels include compounds (e.g., biotin and digoxigenin), that bind to anti-ligands or antibodies labeled with fluorophores, chemiluminescent agents, or enzymes. Alternatively, probes can be conjugated directly to labels such as fluorophores, chemiluminescent agents or enzymes. The choice of label depends on sensitivity required, ease of conjugation with the probe, stability requirements, and available instrumentation.

The probes and primers of the invention can be synthesized and labeled using techniques known to one skilled in the art. Oligonucleotides for use as probes and primers may be chemically synthesized according to the solid phase phosphoramidite triester method described by Beaucage, S. L. and Caruthers, M. H., 1981, Tetrahedron Lett. 22(20): 1859-1862, using an automated synthesizer, as described in Needham-VanDevanter, D. R., et al. 1984, Nucleic Acids Res. 12: 6159-6168. Purification of oligonucleotides can be by either native acrylamide gel electrophoresis or by anion-exchange HPLC, as described in Pearson, J. D. and Regnier, F. E., 1983, J. Chrom. 255:137-149. All of the references cited supra are incorporated herein by reference in their entirety.

In some embodiments, the invention encompasses detecting a nucleic acid encoding an ErbB receptor using a disposable dipstick device such as the one described in WO 00/29112, which is incorporated herein by reference in its entirety, prepared in view of the present disclosure. However, it will be appreciated by one skilled in the art that any device known in the art for the detection of a nucleic acid molecule is within the scope of the methods of the present invention. Briefly, detection of a nucleic acid using the dipstick device disclosed in WO 00/29112 provides one-step detection of a nucleic acid sequence in a sample. Typically, the device is operated by aspirating the sample to be analyzed through a tube into a chamber. Inside the chamber, the sample is prepared by contact with pre-measured and pre-deposited reagents. The sample is subsequently processed by mobilization via capillary action through a membrane having thereon pre-measured and pre-deposited signaling and/or detection reagents for specific detection of one or more nucleic acids of interest in the sample, e.g., a nucleic acid encoding an ErbB-receptor.

4.3.3 Detection of Proteins

The methods and kits of the invention encompass detection and/or quantitation of any one or more ErbB receptor proteins known in the art, including but not limited to ErbB-1, ErbB-2, ErbB-3, and ErbB-4 in a sample obtained from a subject. Any method known to one skilled in the art for the detection and quantitation of an ErbB receptor protein is encompassed within the present invention. ErbB receptor protein sequences useful in the methods and kits of the invention are well known in the art. See, e.g., Section 4.8 below.

ErbB receptor proteins and anti-ErbB antibodies and immunospecific fragments thereof are suitable in the assays of the invention for evaluating the prognosis of a cancer in a subject.

Detection and quantitation of an ErbB gene product encompasses the detection of proteins exemplified herein. Detection of elevated levels of an ErbB gene product in a sample obtained from a subject in accordance with the methods of the invention is generally compared to a standard sample.

In some embodiments, antibodies directed against naturally occurring ErbB proteins may be used in the prognostic methods of the invention. The invention encompasses the use of any standard immunoassay method known to one skilled in the art, including but not limited to Western blot, ELISA, and FACS.

In one embodiment, the invention encompasses use of an immunoassay comprising contacting a sample from a subject with an anti-ErbB antibody or an immunospecific fragment thereof under conditions such that immunospecific binding to the ErbB receptor in the sample can occur, thereby forming an immune complex, and detecting and/or measuring the amount of complex formed. In a specific embodiment, an antibody to an ErbB receptor is used to assay a sample for the presence of the ErbB receptor, wherein an increased level of the ErbB receptor is detected relative to a standard sample.

In some embodiments, the biological sample may be brought in contact with and immobilized onto a solid phase support or a carrier such as nitrocellulose or other solid support capable of immobilizing cells, cell particles or soluble proteins. The support can be washed with suitable buffers followed by treatment with the antibody that selectively or specifically binds to an ErbB receptor protein. The solid phase support can then be washed with buffer to remove unbound antibody. The amount of antibody bound to the solid support can then be detected by conventional means.

“Solid phase support or carrier” as used herein refers to any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

In some embodiments, the anti-ErbB antibody or an immunospecific fragment thereof can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2: 1, Microbiological Associates Quarterly Publication, Walkersville, Md.; Voller, A. et al., 1978, J. Clin. Pathol. 31:507-520; Butler, J. E., 1981, Meth. Enzymol. 73:482; Maggio, E. ed., 1980, Enzyme Immunoassay, CRC Press, Boca Raton, Fla.,; Ishikawa, E. et al., eds., 1981, Enzyme Immunoassay, Kgaku Shoin, Tokyo, all of which are incorporated herein by reference in their entirety). The enzyme bound to the antibody will react with an appropriate substrate, preferably a chromogenic substrate, in such a manner as to produce a chemical moiety which can be detected, for example, by spectrophotometric, fluorimetric or visual means. Enzymes that can be used to detectably label the antibody include but are not limited to malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase, among others. The detection can be accomplished by colorimetric methods that employ a chromogenic substrate for the enzyme. Detection can also be accomplished by visual comparison of the extent of enzymatic reaction of a substrate in comparison with similarly prepared standards.

Detection can also be accomplished using any other method known to one skilled in the art. For example, by radioactively labeling the antibodies or antibody fragments, it is possible to detect ErbB receptor protein through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter, or by autoradiography.

In other embodiments, the invention encompasses labeling the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. In yet other embodiments, the antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The invention further encompasses detectably labeling the antibody by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds for purposes of labeling include, e.g., luciferin, luciferase and aequorin.

The invention also encompasses methods for indirect detection of an ErbB receptor protein. In a specific embodiment, the invention encompasses use of an immunoassay comprising contacting a sample derived from a subject with cancer with an anti-ErbB antibody (primary antibody) or an immunospecific fragment thereof under conditions such that immunospecific binding to the ErbB receptor protein in the sample can occur, thereby forming an immune complex, adding a secondary antibody that is labeled under conditions such that immunospecific binding to the primary antibody occurs and detecting and/or quantitating the amount of complex formed indirectly.

Anti-ErbB antibodies or immunospecific fragments thereof may be used quantitatively or qualitatively to detect an ErbB receptor in a sample. In some embodiments, when the sample is a tissue, the anti-ErbB antibodies or immunospecific fragments thereof may be used histologically, e.g., immunofluorescence or microscopic studies, using common techniques known to one skilled in the art, for in situ detection of an ErbB receptor. In situ detection may be accomplished by preparing a histological specimen from a subject, such as a paraffin embedded section of tissue, e.g., breast tissues, and applying thereto a labeled antibody of the present invention. The antibody (or fragment) is preferably applied by overlaying the labeled antibody (or fragment) onto the biological sample. Through the use of such a procedure, it is possible to determine not only the presence of an ErbB receptor protein but also its distribution in the examined tissue. Using the methods of the present invention, those of ordinary skill will readily perceive that any of a wide variety of histological methods (such as staining procedures) can be modified in order to achieve such in situ detection.

In other embodiments, ligands of an ErbB receptor can be used to quantify the number of receptors in a sample using methods known to one skilled in the art. See e.g., Goodman et al., 1996 eds.; Goodman and Gilman's: The Pharmacological Basis of Therapeutics, New York, McGraw Hill, which is incorporated herein by reference in its entirety. Ligands for ErbB receptors are well known in the art and their use is encompassed within the methods and kits of the invention. For example, neuregulins are known to bind ErbB-2, ErbB-3, and ErbB-4. The neuregulins (NRGs) are cell-cell signaling proteins that are ligands for receptor tyrosine kinases of the ErbB family. The neuregulin family of genes has four members: NRG1, NRG2, NRG3, and NRG4 (see review by Falls, 2003, Exp. Cell. Res. 284(1): 14-30; which is incorporated herein by reference in its entirety). Any nucleotide sequence encoding a neuregulin may be used in connection with the methods and kits of the present invention, including but not limited to human NRG1 with GENBANK Accession No. AY207002; variants of human NRG2 with GENBANK Accession Nos. NM-013985; NM-013984; NM-013983; NM-013982; NM-013981; NM-004883, NRG3, as described in Zhang et al., 1997, Proc. Natl. Acad. Sci. USA. 94: 9562-7; which is incorporated herein by reference in its entirety. Such NRG nucleic acid sequences are useful for recombinant production of NRG proteins by conventional methods, and said proteins can be used as ligands to quantitate ErbB receptors using well known methods.

4.4 Cancers

The prognostic methods of the invention may be useful for any cancer, particularly those involving an aberrant expression of an ErbB receptor protein. As used herein, the term “cancer” carries its ordinary meaning in the art and refers to a neoplasm or tumor resulting from abnormal uncontrolled growth of cells. In some embodiments, cancer refers to a benign tumor that has remained localized. In other embodiments, cancer refers to a malignant tumor that has invaded and destroyed neighboring body structures and spread to distant sites.

Cancers and related disorders that can benefit from the prognostic methods of the invention include but are not limited to the following: Leukemias including but not limited to acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to chronic myelocytic (granulocytic) leukemia, chronic lymphocytic leukemia, hairy cell leukemia, polycythemia vera, lymphomas such as but not limited to Hodgkin's disease, and non-Hodgkin's disease, multiple myelomas such as but not limited to smoldering multiple myeloma, nonsecretory myeloma, osteosclerotic myeloma, plasma cell leukemia, solitary plasmacytoma and extramedullary plasmacytoma, Waldenström's macroglobulinemia, monoclonal gammopathy of undetermined significance, benign monoclonal gammopathy, heavy chain disease, bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma, brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma, breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer, adrenal cancer including but not limited to pheochromocytom and adrenocortical carcinoma, thyroid cancer such as but not limited to papillary or follicular thyroid cancer, medullary thyroid cancer and anaplastic thyroid cancer, pancreatic cancer including but not limited to insulinoma, gastrinoma, glucagonoma, vipoma, somatostatin-secreting tumor, and carcinoid or islet cell tumor, pituitary cancers including but not limited to Cushing's disease, prolactin-secreting tumor, acromegaly, and diabetes insipius, eye cancers including but not limited to ocular melanoma such as iris melanoma, choroidal melanoma, cilliary body melanoma, and retinoblastoma, vaginal cancers including but not limited to squamous cell carcinoma, adenocarcinoma, and melanoma; vulvar cancer, including but not limited to, squamous cell carcinoma, melanoma, adenocarcinoma, basal cell carcinoma, sarcoma, and Paget's disease cervical cancers including but not limited to squamous cell carcinoma, and adenocarcinoma, uterine cancers including but not limited to endometrial carcinoma and uterine sarcoma, ovarian cancers including but not limited to ovarian epithelial carcinoma, borderline tumor, germ cell tumor, and stromal tumor, esophageal cancers including but not limited to squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma, stomach cancers including but not limited to adenocarcinoma, fungating (polypoid), ulcerating, superficial spreading, diffusely spreading, malignant lymphoma, liposarcoma, fibrosarcoma, and carcinosarcoma, colon cancers, rectal cancers, liver cancers including but not limited to hepatocellular carcinoma and hepatoblastoma, gallbladder cancers including but not limited to adenocarcinoma, cholangiocarcinomas including but not limited to papillary, nodular, and diffuse, lung cancers including but not limited to non-small cell lung cancer, squamous cell carcinoma (epidermoid carcinoma), adenocarcinoma, large-cell carcinoma and small-cell lung cancer, testicular cancers including but not limited to germinal tumor, seminoma, anaplastic, classic (typical), spermatocytic, nonseminoma, embryonal carcinoma, teratoma carcinoma, choriocarcinoma (yolk-sac tumor), prostate cancers including but not limited to adenocarcinoma, leiomyosarcoma, and rhabdomyosarcoma; renal cancers; oral cancers including but not limited to squamous cell carcinoma, basal cancers, salivary gland cancers including but not limited to adenocarcinoma, mucoepidermoid carcinoma, and adenoidcystic carcinoma, pharynx cancers including but not limited to squamous cell cancer, and verrucous, skin cancers including but not limited to basal cell carcinoma, squamous cell carcinoma and melanoma, superficial spreading melanoma, nodular melanoma, lentigo malignant melanoma, acral lentiginous melanoma, kidney cancers including but not limited to renal cell cancer, adenocarcinoma, hypernephroma, fibrosarcoma, and transitional cell cancer (renal pelvis and/or uterer), Wilms' tumor, bladder cancers including but not limited to transitional cell carcinoma, squamous cell cancer, adenocarcinoma, and carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The methods and kits of the invention are also useful in the prognosis of a variety of cancers or other abnormal proliferative diseases, including (but not limited to) the following: carcinoma, including that of the bladder, breast, colon, kidney, liver, lung, ovary, pancreas, stomach, prostate, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, and Burketts lymphoma; hematopoietic tumors of myeloid lineage including acute and chronic myelogenous leukemias and promyelocytic leukemia, tumors of mesenchymal origin including fibrosarcoma and rhabdomyoscarcoma; other tumors including melanoma, seminoma, tetratocarcinoma, neuroblastoma and glioma, tumors of the central and peripheral nervous system, including astrocytoma, neuroblastoma, glioma, and schwannomas, tumors of mesenchymal origin, including fibrosafcoma, rhabdomyoscarama, and osteosarcoma, and other tumors, including melanoma, xenoderma pegmentosum, keratoactanthoma, seminoma, thyroid follicular cancer and teratocarcinoma. In specific embodiments, the prognostic methods of the invention are useful in malignancy or dysproliferative changes (such as metaplasias and dysplasias), or hyperproliferative disorders, in the ovary, bladder, breast, colon, lung, skin, pancreas, or uterus. In other specific embodiments, the prognostic methods of the invention are useful in sarcoma, melanoma, or leukemia.

4.5 Subjects

The diagnostic and prognostic methods and kits of the invention are useful in any subject, including mammals such as companion animals, most preferably humans. Preferably, the subject has been diagnosed with an ErbB-1 positive tumor. In a specific embodiment, the subject may have been treated with any standard therapy known to one skilled in the art for the treatment and/or prevention and/or management of cancer, particularly a cancer associated with an aberrant expression and/or activity of an ErbB receptor. Such treatment regimens are known in the art, several of which are described in Noonberg et al., 2000, Drugs 59(4): 753-767, which is incorporated herein by reference in its entirety. In a preferred embodiment, the subject has previously been treated with a chemotherapy regimen specific for an ErbB-1 positive tumor. The chemotherapy regimen can include any chemotherapy treatment known in the art for treatment of cancer, particularly a cancer associated with aberrant expression and/or activity of an ErbB receptor including but not limited to treatment with a chemotherapeutic agent directed at the ErbB signaling pathway, such as IMC-225 (an antibody that binds ErbB-1 and is believed to block EGF-induced autophosphorylation), or ZD1839 (a quinizalone derivative that is a selective reversible inhibitor of ErbB-1 tyrosine kinase activity).

The subject may have been treated with any chemotherapeutic agent (or “anti-cancer agent” or “anti-tumor agent” or “cancer therapeutic”) which, as used herein, refers to any molecule or compound that assists in the treatment of tumors or cancer. Examples of such agents include but are not limited to cytosine arabinoside, taxoids (e.g., paclitaxel, docetaxel), anti-tubulin agents (e.g., paclitaxel, docetaxel, epothilone B, or its analogues), macrolides (e.g., rhizoxin) cisplatin, carboplatin, adriamycin, tenoposide, mitozantron, discodermolide, eleutherobine, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), anti-metabolites (e.g., methotrexate, 6-mercaptopurine, 6-thioguanine, cytarabine, flavopiridol, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, colchine, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gamncitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath-1, straplozocin, thioguanine, tretinoin, vinorelbine, or any fragments, family members, or derivatives, or analogs thereof. Further example of chemotherapeutic agents may be found in standard texts. See, e.g., Manual of Clinical Oncology, Dennis A. Casciato and Barry A. Lowitz, e.d., 4^{th} edition, Jul. 15, 2000, Little, Brown and Company, U.S.

In a specific embodiment, the chemotherapeutic agent is a quinazoline derivative directed at inhibiting the tyrosine kinase activity of an ErbB receptor. Such compounds are known in the art and include such compounds as ZD1839 (Zeneca Pharmaceuticals); CP-358,774 (Pfizer, Groton, Conn.); and CGP 59326A (Novartis, Basel, Switzerland) (For a review see Woodburn, 1999, Pharmacol. Ther. 82(2-3): 241-50; de Bono et al., 2002, Trends in Mol. Med, 8(4): S19-26; Noonberg et al., 2000, Drugs 59(4): 753-767; all of which are incorporated herein by reference in their entirety).

In yet another specific embodiment, the subject may be treated with Trastuzumab (Herceptin; Genentech) which targets ErbB2 and is available commercially.

In other embodiments, the invention encompasses any small molecule inhibitor of an ErbB receptor tyrosine kinase activity that is currently in clinical development, including but not limited to OSI-774 (OSI/Genentech), which is a quinazoline derivative, and competitively inhibits ATP-binding of ErbB-1; PKI 116 (Novartis) which is a pyrrolopyrimidine and competitively inhibits ATP binding of ErbB-1; GW2016 (Glaxo Smithkline), which is a quinazoline derivative and competitively inhibits ATP binding of ErbB-1 and ErbB-2; EKB-569 (Genetics Institute, Wyeth-Ayerst), which is a 3-cyanoquinoline derivative and irreversibly binds ErbB1 at the ATP binding site and is reported to inhibit growth of ErbB-1 and ErbB-2 positive tumors and irreversibly blocks tyrosine kinase activity of ErbB receptors; and CI-1033 (Pfizer), which is a quinozoline derivative and competitively inhibits ATP binding site of all ErbB receptors, particularly ErbB-1 and ErbB-2.

4.6 Kits

Kits for performing a method of the invention are also provided. In one embodiment, the invention provides a kit comprising in one or more containers an anti-ErbB antibody or an immunospecific fragment thereof, and optionally a labeled binding partner to the antibody or a fragment thereof. Alternatively, the anti-ErbB antibody can be labeled with a detectable marker, (e.g., a chemiluminescent, enzymatic, fluorescent, or radioactive moiety).

In another embodiment, a kit is provided that comprises in one or more containers a nucleic acid probe or probes capable of hybridizing to an ErbB receptor-encoding mRNA. In a specific embodiment, a kit can comprise in one or more containers a pair of primers (e.g., each in the size range of 6-30 nucleotides) that are capable of priming amplification, e.g., by PCR (see, e.g., Innis et al., 1990, PCR Protocols, Academic Press, Inc., San Diego, Calif.; Erlich, ed., 1989, PCR Technology, Principles and Applications for DNA Amplification, Stockton Press, New York; Erlich et al., 1991, Science 252: 1643-1651), ligase chain reaction (EP 320,308), use of b-replicase, cyclic probe reaction, or other methods known in the art under appropriate conditions of at least a portion of an ErbB receptor encoding nucleic acid.

In another specific embodiment, a kit is provided that consists in one or more containers a nucleic acid probe or probes capable of hybridizing to an ErbB receptor-encoding mRNA such that no amplification of the ErbB receptor-encoding mRNA is needed.

A kit can optionally further comprise in a container a predetermined amount of a purified ErbB receptor protein or a nucleic acid encoding an ErbB receptor for use as a standard or control useful in quantifying the amount of ErbB receptor protein or mRNA. Each kit may also include printed instructions and/or a printed label describing the practicing of the invention in accordance with one or more of the embodiments described herein. Kit containers may optionally be sterile containers.

4.7 Nucleic Acids Encoding ErbB Receptors

The methods of the invention may use any nucleic acid encoding an ErbB receptor, including but not limited to ErbB-1, ErbB-2, ErbB-3, and ErbB-4, as a proxy for determining an ErbB receptor level. A nucleic acid is intended to include DNA molecules (e.g., cDNA, genomic DNA), RNA molecules (e.g., hnRNA, pre-mRNA, mRNA) and DNA or RNA analogs (e.g., peptide nucleic acids) generated using techniques known to one skilled in the art. The nucleic acid measured as a proxy for an ErbB receptor level can be single-stranded or double stranded.

For example, but not by way of limitation, nucleotide sequences for use in the methods and kits of the invention may include all or a portion of any of the following: the nucleotide sequence of human ErbB-1, as determined from placental and A431 carcinoma cells (see Ulrich et al., 1984, Nature 309:418-425); the nucleotide sequence of rat ErbB-1 with GENBANK accession number NM-031507; the nucleotide sequence of human ErbB-2, as determined from a human fetal DNA library (Coussens et al., 1985, Science 230: 1132-9); nucleotide sequences of any of exons 1-7 of human ErbB-2 with GENBANK accession numbers AH001455, M11762, M11763, M11764, M11765, M11766, and M11767, respectively; the nucleotide sequence of ErbB-3 determined from a human carcinoma cell line (Plowman et al., 1990, Proc. Natl. Acad. Sci. USA. 87: 4905-9); the nucleotide sequence of gallus ErbB-4 with GENBANK accession number AF041792; and a nucleotide sequence of ErbB-4 from a murine melanoma cell line (Plowman et al., 1993, Proc. Natl. Acad. Sci. USA. 90: 1746-50). All nucleotide sequences of the references cited supra are incorporated herein by reference in their entirety.

Generally, any ErbB receptor nucleic acid known in the art may be useful in the methods and kits of the invention. Such nucleic acids generally encode at least a portion of an ErbB receptor, e.g., ErbB-1, ErbB-2, ErbB-3, or ErbB-4, or have a sequence that hybridizes to an ErbB receptor-encoding nucleic acid under hybridizing conditions, as described herein.

In one embodiment, the methods of the invention may use a coding sequence or a 5′ or 3′ untranslated region of a nucleic acid encoding an ErbB receptor or a fragment thereof as a probe, including naturally occurring and non-naturally occurring variants. A non-naturally occurring variant is one that is engineered by man (e.g., a peptide nucleic acid probe). In the methods of the invention wherein an ErbB receptor or an mRNA encoding an ErbB receptor in a sample from a subject is detected or measured, naturally occurring gene products are detected, including but not limited to wild-type gene products as well as mutants, allelic variants, splice variants, polymorphic variants, etc. In general, variants will be highly homologous to the wild-type gene product encoding an ErbB receptor, e.g., having at least 90%, 95%, 98% or 99% amino acid sequence identity (as determined by standard algorithms known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul et al., 1990 J. Mol. Biol. 215: 403-410).

ErbB receptor variants to be used as probes may be encoded by a nucleic acid which is hybridizable under stringent conditions to a nucleic acid encoding an ErbB receptor. Nucleic acid hybridization methods are well known in the art (see, e.g., Sambrook et al., 2001 Molecular Cloning, A Laboratory Manual, 3^{rd} ed., Cold Spring Harbor Laboratory Press, Cold Spring Harbor, N.Y.; Ausubel et al., eds., 1994-1997, in the Current Protocols in Molecular Biology: Series of laboratory technique manuals, John Wiley and Sons, Inc.; Shilo and Weinberg, 1981, Proc. Natl. Acad. Sci. U.S.A. 78, 6789-92; Dyson, 1991 Essential Molecular Biology: A Practical Approach, vol. 2, T. A. Brown, ed., 111-156, Press at Oxford University Press, Oxford, UK). The term “stringent conditions” refers to the ability of a first polynucleotide molecule to hybridize, and remain bound to a second filter-bound polynucleotide molecule in 0.5 M NaHPO₄, 7% sodium dodecyl sulfate (SDS), 1 mM EDTA at 65° C., followed by washing in 0.2×SSC/0.1% SDS at 42° C. (see Ausubel et al. (eds.), 1989, Current Protocols in Molecular Biology, Vol. I, Green Publishing Associates, Inc., and John Wiley & Sons, Inc., New York, at p. 2.10.3). In specific embodiments, the variants being detected or measured comprise (or, if nucleic acids, encode) not more than 1, 2, 3, 4, 5, 10, 15 or 20 point mutations (substitutions) relative the wild-type sequence.

An isolated nucleic acid probe encoding an ErbB receptor family member, e.g., ErbB-1, ErbB-2, ErbB-3, or ErbB-4 or a portion thereof, can be obtained by any method known in the art, e.g., from a deposited plasmid, by PCR amplification using synthetic primers hybridizable to the 3′ and 5′ ends of the sequence, and/or by cloning from a cDNA or genomic library using standard screening techniques, or by polynucleotide synthesis. Use of such probes for detection and quantitation of specific sequences is well known in the art. See e.g., Erlich, e.d., 1989, PCR Technology Principles and Applications for DNA Amplification, Macmillan Publishers Ltd., England; Sambrook et al, Molecular Cloning: A Laboratory Manual, 3^{rd} ed., Cold Spring Harbor Laboratory Press, Cold Spring Harbor, N.Y., 2001.

In some embodiments, the methods of the invention may use a gene coding sequence, e.g., cDNA, of an ErbB receptor, including but not limited to, ErbB-1, ErbB-2, ErbB-3, and ErbB-4, which preferably hybridizes under stringent conditions as described above to at least about 6, preferably about 12, most preferably about 18 or more consecutive nucleotides of the gene coding sequence of an ErbB receptor protein, useful for the detection of an ErbB receptor protein for the prognosis of cancer as described herein.

Using all or a portion of a nucleic acid sequence encoding an ErbB receptor protein, such as those exemplified herein as a hybridization probe, full length nucleic acid molecules encoding an ErbB receptor protein can be quantitated using standard hybridization techniques (see, e.g., Sambrook et al., Molecular Cloning: A Laboratory Manual, 3^{rd} ed., Cold Spring Harbor Laboratory, Cold Spring Harbor Laboratory Press, Cold Spring Harbor, N.Y., 2001) for use in the methods of the invention, i.e., as a proxy for an ErbB receptor level.

The ErbB receptor sequences used in the methods of the invention are preferably human sequences. However, homologs of the human ErbB receptor isolated from other animals can also be used in the methods of the invention as a proxy for an ErbB receptor level, particularly where the subject is a non-human animal. Thus, the invention also includes the use of ErbB receptor homologs identified from non-human animals such as non-human primates, rats, mice, farm animals including but not limited to cattle, horses, goats, sheep, pigs, etc., household pets including but not limited to cats, dogs, etc., in the methods of the invention.

The methods of the invention may use fragments of any of the nucleic acids disclosed herein in any of the methods of the invention. A fragment preferably comprises at least 10, 20, 50, 100, or 200 contiguous nucleotides of a sequence described herein.

Standard recombinant DNA techniques known in the art may be used to provide an ErbB receptor protein or a nucleic acid encoding an ErbB receptor protein, or a fragment thereof, for use in the methods and kits of the invention. In some embodiments, in order to provide an ErbB protein or nucleic acid as a standard, the corresponding nucleotide sequence encoding an ErbB protein of interest can be cloned. For a review of PCR technology and cloning strategies which may be used in accordance with the invention, see, e.g., PCR Primer, 1995, Dieffenbach et al., ed., Cold Spring Harbor Laboratory Press; Sambrook et al., 2001, supra.

4.8 ErbB Receptor Proteins

The present invention provides for the use of ErbB receptor proteins, including but not limited to ErbB-1, ErbB-2, ErbB-3, and ErbB-4 polypeptides, or fragments thereof, for the generation of antibodies for methods of the invention. ErbB receptor polypeptides and fragments can also be used as protein abundance or activity standards in the methods of the invention.

For example, but not by way of limitation, amino acid sequences of ErbB receptors include that of human ErbB-1 from placental and A431 carcinoma cell lines (Uhlrich et al., 1984, Nature 309: 418-25); or of human ErbB-2 cloned from a human fetal cDNA library (Coussens et al., 1985, Science 230: 1132-9); or of human ErbB-3 from a human carcinoma cell line (Plowman et al., 1990, Proc. Natl. Acad. Sci. USA. 87: 4905-9); or of the amino acid sequence of ErbB-4 corresponding to a cDNA clone isolated from a murine melanoma cell line (Plowman et al., 1993, Proc. Natl. Acad. Sci. USA. 90: 1746-50). The amino acid sequences cited in the above-identified references are incorporated herein by reference in their entirety.

In some embodiments, the ErbB receptor protein comprises an amino acid sequence that exhibits at least about 65%, 70%, 75%, 80%, 85%, 90%, 95%, or 99% sequence similarity to the amino acid sequence of any of ErbB-1, ErbB-2, ErbB-3, or ErbB-4. Algorithms for determining percent identity between two protein sequences are well known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul et al., 1990 J. Mol. Biol. 215: 403-410.

In a specific embodiment, proteins are provided consisting of or comprising a fragment of an ErbB receptor protein consisting of at least ten contiguous amino acids. In another embodiment, the fragment consists of or comprises at least 20, 30, 40, or 50 contiguous amino acids from an ErbB receptor for use, for example, in raising antibodies. Such fragments can also be useful, for example, as standards or controls in the methods and kits of the invention.

A variety of host-expression vector systems may be utilized to express ErbB receptor proteins or fragments for use in the methods of the invention. Such host-expression systems are well known and provide the necessary means by which a protein of interest may be produced and subsequently purified. Examples of host-expression vector systems that may be used in accordance with the invention are: bacterial cells (e.g., E. coli, B. subtilis) transformed with recombinant bacteriophage DNA, plasmid DNA or cosmid DNA expression vectors containing an ErbB receptor nucleic acid coding sequence, yeast cells (e.g., Saccharomyces, Pichia) transformed with a recombinant yeast expression vector containing the ErbB receptor coding sequence; insect cells infected with a recombinant virus expression vector (e.g., baculovirus) containing the ErbB receptor coding sequence; plant cells infected with a recombinant virus expression vector (e.g., cauliflower mosaic virus, CaMV; tobacco mosaic virus, TMV) or transformed with a recombinant plasmid expression vector (e.g., Ti plasmid) containing the ErbB receptor coding sequence; or mammalian cells (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the ErbB receptor being expressed. For example, when a large quantity of such a protein is to be produced for raising antibodies, vectors that direct the expression of high levels of protein products that are readily purified may be desirable. Such vectors include but are not limited to the E. coli expression vector pUR278 (Ruther et al., 1983, EMBO J. 2:1791), in which the ErbB receptor coding sequence can be ligated into the vector in-frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors can also be used to express foreign polypeptides as fusion proteins with glutathione S-transferase (GST). In general, such fusion proteins are soluble and can easily be purified from lysed cells by adsorption and binding to a column comprising of glutathione-agarose beads followed by elution in the presence of free glutathione. The pGEX vectors are designed to include, e.g., thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The ErbB receptor coding sequence can be cloned individually into non-essential regions (for example the polyhedrin gene) of the virus and placed under control of an AcNPV promoter (for example the polyhedrin promoter). Successful insertion of an ErbB receptor coding sequence will result in inactivation of the polyhedrin gene and production of non-occluded recombinant virus (i.e., virus lacking the proteinaceous coat coded for by the polyhedrin gene). These recombinant viruses can be used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems can be utilized. In cases where an adenovirus is used as an expression vector, the ErbB receptor coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing ErbB receptor in infected hosts (see, e.g., Logan & Shenk, 1984, Proc. Natl. Acad. Sci. USA 81:3655). Specific initiation signals may also be required for efficient translation of inserted ErbB receptor coding sequences. These signals include the ATG initiation codon and adjacent sequences. In cases where an entire ErbB receptor family member gene, including its own initiation codon and adjacent sequences, is inserted into the appropriate expression vector, no additional translational control signals may be needed. However, in cases where only a portion of the ErbB receptor coding sequence is inserted, exogenous translational control signals, including, if necessary, the ATG initiation codon, must be provided. These exogenous translational control signals and initiation codons can be of a variety of origins, both natural and synthetic. Furthermore, the initiation codon must be in phase with the reading frame of the desired coding sequence to ensure correct translation of the entire insert. The efficiency of expression may be enhanced by the inclusion of appropriate transcription enhancer elements, transcription terminators, etc. (see Bittner et al., 1987, Methods in Enzymol. 153: 516).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, COS, MDCK, 293, 3T3, WI38, and in particular, breast cancer cell lines such as, for example, BT483, Hs578T, HTB26, BT20 and T47D, and normal mammary gland cell lines such as, for example, CRL7030 and Hs578Bst.

For long-term, high-yield production of recombinant proteins, stable expression is preferred. For example, cell lines that stably express the ErbB receptor gene product can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker.

Following introduction of the foreign DNA, engineered cells can be allowed to grow for 1-2 days in an enriched media, and then can be switched to a selective media. A selectable marker in a recombinant construct, such as a plasmid, can confer resistance to the selective media, allow cells to stably integrate the plasmid into their chromosomes, and grow to form foci which, in turn, can be cloned and expanded into cell lines. This method can advantageously be used to engineer cell lines that stably express the ErbB receptor gene product. Such engineered cell lines can be particularly useful in screening and evaluating compounds that affect the endogenous activity of the ErbB receptor family member gene product.

A number of selection systems including but not limited to the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., 1980, Proc Natl. Acad. Sci. USA 77: 3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

4.9 Antibodies to ErbB Receptors, Derivatives and Analogs

The methods and kits of the invention encompass use of anti-ErbB receptor antibodies or fragments thereof that specifically recognize one or more epitopes of an ErbB receptor protein, e.g., ErbB-1, ErbB-2, ErbB-3, or ErbB-4. Accordingly, any ErbB receptor protein, derivative, or fragment can be used as an immunogen to generate antibodies that immunospecifally bind an ErbB receptor protein. Such antibodies and fragments can be used in the detection and quantitation of an ErbB receptor in a sample to carry out any of the methods of the invention as disclosed herein.

Such antibodies can include but are not limited to polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. In a specific embodiment, antibodies to human ErbB-2, -3 or -4 receptor protein are used.

Described herein are general methods for the production of antibodies or immunospecific fragments thereof. Any of such antibodies or fragments can be produced by standard immunological methods or by recombinant expression of nucleic acid molecules encoding the antibody or an immunospecific fragment thereof in an appropriate host organism.

For the production of antibodies against an ErbB receptor, any of various host animals can be immunized by injection with an ErbB receptor gene product, or a portion thereof. Such host animals can include but are not limited to rabbits, mice, and rats. Various adjuvants can be used to increase the immunological response depending on the host species, including but not limited to Freund's (complete or incomplete), mineral gels such as aluminum hydroxide, surface active substances such as lysolecithin, pluronic polyols, polyanions, peptides, oil emulsions, keyhole limpet hemocyanin, dinitrophenol or potentially useful human adjuvants such as BCG (bacille Calmette-Guerin) and Corynebacterium parvum.

Anti-ErbB receptor monoclonal antibodies are preferred for use in the methods and kits of the invention. Monoclonal antibodies can be obtained by any technique that provides for the production of antibody molecules by continuous cell lines in culture. These include but are not limited to the hybridoma technique of Kohler and Milstein, (1975, Nature 256: 495; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026), and the EBV-hybridoma technique (Cole et al., 1985, Monoclonal Antibodies And Cancer Therapy, Alan R. Liss, Inc., pp. 77). Such antibodies can be of any immunoglobulin class, including IgG, IgM, IgE, IgA, IgD, and any subclass thereof. The hybridoma producing the mAb of this invention can be cultivated in vitro or in vivo.

Techniques developed for the production of “chimeric antibodies” (Morrison et al., 1984, Proc. Natl. Acad. Sci. 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used in preparing antibodies useful in the present invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for an ErbB receptor protein. While often designed to be therapeutic, such chimeric antibodies can be useful to quantitate an ErbB receptor level according to the methods of the invention.

Further, humanized antibodies can be used in the methods and kits of the invention. Briefly, humanized antibodies are antibody molecules from non-human species having one or more hypervariable regions or complementarity determining regions (CDRs) from the non-human species and framework regions from a human immunoglobulin molecule. The extent of the framework region and Cars have been precisely defined (see, “Sequences of Proteins of Immunological Interest”, Kabat, E. et al., U.S. Department of Health and Human Services (1983)). Examples of techniques that have been developed for the production of humanized antibodies are known in the art and useful within the scope of the present invention. (See, e.g., Queen, U.S. Pat. No. 5,585,089 and Winter, U.S. Pat. No. 5,225,539). Humanized antibodies are typically developed as therapeutic agents. However, such antibodies can also be used in the methods and kits of the present invention, as they can be used to quantitate an ErbB receptor level in accordance with the instant invention.

Phage display technology can be used to increase the affinity of an antibody to an ErbB receptor gene product. This technique can be useful in obtaining higher affinity antibodies to an ErbB receptor gene product, which could be used for the diagnosis and prognosis of a subject with cancer according to the present invention. The technology, referred to as affinity maturation, employs mutagenesis or CDR walking and re-selection using the ErbB receptor gene product antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones, each of which differs by a single amino acid alteration in a single CDR, and contain variants representing each possible amino acid substitution for each CDR residue. Mutants with increased binding affinity for the antigen can be screened by contacting the immobilized mutants with labeled antigen. Any screening method known in the art can be used to identify mutant antibodies having increased avidity to the antigen (e.g., ELISA) (see Wu et al., 1998, Proc Natl. Acad. Sci. USA 95:6037; Yelton et al., 1995, J. Immunology 155:1994). CDR walking that randomizes the light chain may also be useful (see Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334: 544) can be adapted to produce single chain antibodies against ErbB receptor gene products. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., 1988, Science 242:1038).

Antibody fragments that recognize specific epitopes can be generated by known techniques. Such fragments can be used for quantitating an ErbB receptor gene product according to any available method known in the art. For example, such fragments include but are not limited to: F(ab′)₂ fragments, which can be produced by pepsin digestion of the antibody molecule; and Fab fragments, which can be generated by reducing the disulfide bridges of the F(ab′)₂ fragments; Fab fragments, which can be generated by treating the antibody molecule with papain and a reducing agent; and Fv fragments. Alternatively, Fab expression libraries can be constructed (Huse et al., 1989, Science 246:1275-1281) to allow rapid and easy identification of monoclonal Fab fragments having the desired specificity.

A molecular clone of an antibody to an antigen of interest can be prepared by techniques known to one skilled in the art. Recombinant DNA methodology (see e.g., Maniatis et al., 1982, Molecular Cloning, A Laboratory Manual, Cold Spring Harbor Laboratory, Cold Spring Harbor, N.Y.) can be used to construct nucleic acid sequences that encode a monoclonal antibody molecule, or an immunospecific fragment thereof.

Antibody molecules can be purified by well-known techniques, e.g., immunoabsorption or immunoaffinity chromatography, chromatographic methods such as HPLC (high performance liquid chromatography), or a combination thereof.

In the production of antibodies, screening for the desired antibody can be accomplished by techniques known in the art, e.g., ELISA (enzyme-linked immunosorbent assay). For example, to select antibodies that recognize a specific domain of an ErbB receptor, generated hybridomas can be assayed for a product that binds to an ErbB receptor fragment containing such domain.

The foregoing antibodies can be used to quantify an ErbB receptor protein, e.g., to measure levels thereof in appropriate samples, in the methods and kits of the invention.

The methods of antibody production employed herein include those described in Harlow and Lane (Harlow, E. and Lane, D., 1988, and later editions, Antibodies: A Laboratory Manual, Cold Spring Harbor Laboratory Press, Cold Spring Harbor, N.Y.), which is incorporated herein by reference in its entirety.

Any antibody directed to one or more epitopes of an ErbB receptor can be used in the methods and kits of the invention. A non-limiting example of ErbB-specific antibodies are IMC-225, which is an ErbB-1 specific antibody (also known as Cetuximab®, ImClone Systems, New York; see Goldstein et al., 1995, Clin. Cancer Res. 1: 1311-8; Prewett et al., 1996, J. Immunother. Tumor Immunol. 19: 419-27; Fan et al., 1994, J. Biol. Chem. 269: 27585-602; Baselga, 2001, Eur. J. Cancer 37: S16-22; all of which are incorporated herein by reference in their entirety); and ABX-EGF, which is an ErbB-1 specific antibody (Abgenix, Fremont, Calif.; a human monoclonal antibody; Yang et al., 2000, Proc. Am. Soc. Clin. Oncol. 19: 48; Sampson et al., 2000, Proc. Natl. Acad. Sci. USA 97: 7503-8; both of which are incorporated herein by reference in their entirety); and Trastuzumab, which is an ErbB-2 specific antibody (for a review see, Baselga et al., 2001, Semin. Oncol. 28: 4-11; Hancock et al., 1991, Cancer Res. 51: 4575-80; which is incorporated herein by reference in its entirety). For a review of ErbB-specific antibodies see Ciardello et al., 2002, Clin. Cancer Res. 7: 2958-70; Noonberg et al., 2000, Drugs, 753-767; and de Bono et al., 2002, Trends in Mol. Med. 8(4): S19-26; Woodburn, 1999, Pharmacol. Ther. 82: 241-50; all of which are incorporated herein by reference in their entirety. Additionally, commercially available ErbB receptor antibodies can be used in accordance with the instant invention, for example those available from Upstate USA, Inc. Charlottsville, Va.; http://www.upstate.com

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout this application various publications are cited. Their contents are hereby incorporated by reference into the present application in their entireties for all purposes.

1. (canceled)

2. A method for determining the prognosis of a cancer in a subject, which subject has been previously treated with a radiotherapy or a chemotherapy regimen for an ErbB-1 positive tumor, said method comprising:
(a) measuring a level of at least one ErbB receptor in a sample obtained from the subject during a period of remission; and
(b) comparing the level measured in step (a) to a standard level,
wherein elevation of the measured level of at least one ErbB receptor relative to the standard level indicates that the subject is at an increased risk for metastasis, recurrence or relapse of the cancer.

3. (canceled)

4. The method of claim 2, wherein the ErbB receptor measured in step (a) is ErbB-1, ErbB-2, ErbB-3 or ErbB-4, or a combination thereof.

5-7. (canceled)

8. The method of claim 2, wherein the cancer is selected from the group consisting of non-small-cell lung cancer, breast cancer, head and neck cancer, prostate cancer, bladder cancer, ovarian cancer, colorectal cancer, and glioblastoma.

9. (canceled)

10. The method of claim 2, wherein measuring a level of an ErbB receptor is carried out using an ErbB receptor probe.

11. The method of claim 2, wherein measuring a level of an ErbB receptor comprises measuring an ErbB receptor related activity.

12. The method of claim 10, wherein the ErbB receptor probe is selected from the group consisting of a nucleic acid, a protein and a small molecule.

13. The method of claim 12, wherein the protein is an antibody or a fragment thereof.

14. The method of claim 12, wherein the protein is an ErbB receptor ligand or a fragment thereof.

15. The method of claim 13, wherein the antibody is a monoclonal antibody.

16-17. (canceled)

18. The method of claim 2, wherein measuring a level of an ErbB receptor is performed at least quarterly, at least bimonthly, at least monthly, at least biweekly, at least weekly, at least every three days or at least daily.

19. (canceled)

20. (canceled)

21. (canceled)

22. A method for improving the effectiveness of cancer treatment in a subject, which subject has been previously treated with a treatment regimen so as to achieve remission, said method comprises:
(a) measuring a level of at least one ErbB receptor in a sample obtained from the subject during a period of remission; and
(b) comparing the level measured in step (a) to a standard level, wherein elevation of the measured level of at least one ErbB receptor relative to the standard level indicates that the subject is in need of additional treatment, so as to improve the effectiveness of the cancer treatment.

23-26. (canceled)

27. The method of claim 22, wherein the subject has been previously treated with a therapy regimen for an ErbB-1 positive tumor.

28-29. (canceled)

30. The method of claim 22, wherein the ErbB receptor measured is ErbB-2, ErbB-3 or ErbB-4, or a combination thereof.

31-33. (canceled)

34. The method of claim 22, wherein the cancer is selected from the group consisting of non-small-cell lung cancer, breast cancer, head and neck cancer, prostate cancer, bladder cancer, ovarian cancer, colorectal cancer, and glioblastoma.

35. (canceled)

36. The method of claim 22, wherein measuring a level of an ErbB receptor is carried out using an ErbB receptor probe.

37-46. (canceled)
